(19) United States
(12) Patent Application Publication
(10) Pub. No.: US 2021/0361814 A1
(43) Pub. Date: Nov. 25, 2021

(54) Disinfectant Fogger

(71) Applicant: Michelle Gordon, Saint Mary's, WV (US)

(72) Inventor: Michelle Gordon, Saint Mary's, WV (US)

(21) Appl. No.: 16/950,494

(22) Filed: Nov. 17, 2020

Related U.S. Application Data
(60) Provisional application No. 63/028,867, filed on May 22, 2020.

(51) Int. Cl. A61L 9/14; A61L 9/013; A61L 9/012

(52) U.S. Cl. CPC A61L 9/14; A61L 2101/46; A61L 9/012; A61L 9/013

(57) ABSTRACT

This present invention relates to a novel disinfectant fogger device and a disinfecting solution contained therein for disinfecting or sanitizing an enclosed space. The disinfecting solution is intended to kill germs, bacteria, viruses and other harmful microbes. The disinfectant fogger device is comprised of an aerosol container, the disinfecting solution, and a lock top nozzle that is repositionable from a first position to a second position by depressing the nozzle and then twisting it in a select clockwise or counterclockwise direction. Once activated, the pressurized disinfecting solution is released into the air, thereby killing germs, bacteria, viruses and other harmful microbes from all surfaces in the enclosed space.

### CROSS-REFERENCE TO RELATED APPLICATION

The present application claims priority to, and the benefit of, U.S. Provisional Application No. 63/028,867, which was filed on May 22, 2020 and is incorporated herein by reference in its entirety.

### BACKGROUND

The present invention relates generally to the field of disinfecting devices. More specifically, the present invention relates to a disinfectant fogger device for disinfecting airborne bacteria, viruses, germs and fungi within an enclosed space, such as a room, a house, an office building, etc. The disinfectant fogger device of the present invention is comprised of an aerosol canister that contains a disinfectant solution, at least one disinfectant spray opening that releases the disinfectant in a desired direction, a lock top that is activated by twisting and pushing downwards on the canister, thereby forming a push and twist type nozzle that initiates the dispensing of the disinfectant solution. More specifically, to use the disinfectant fogger device of the present invention, the user will push downward on the nozzle and twist the same, such as in a clockwise or counter-clockwise direction, which will in turn cause the disinfectant solution to be expelled from the aerosol canister through one or more openings in the nozzle. As the disinfectant solution is expelled through the spray openings, it is released in an aerosol mist that is sent in an upward direction (i.e., away from the aerosol can which may be positioned on a table of floor) or in a 360 degree pattern to disinfect the entire room, area and/or other enclosed space at the time of dispensing. Accordingly, the present specification makes specific reference thereto. However, it is to be appreciated that aspects of the present invention are also equally amenable to other like applications, devices and methods of manufacture.

Bacterial, fungal, viral and microbial contamination of rooms, offices, restaurants, schools and other confined spaces has long been a source of infection and diseases for humans. Bacteria, viruses, and other disease-causing microbes adhere to surfaces after contact with humans, animals, foods, etc., and also linger in the air within a room after being discharged by a person sneezing, coughing, laughing and even speaking. In this manner, humans have spread infectious diseases among each other since time immemorial including, without limitation, the common cold, influenza, rotavirus, hepatitis A, tuberculosis, conjunctivitis, staphylococcal bacterial infections, strep throat and other streptococcal bacterial infections, as well as the ongoing Covid-19 pandemic. Accordingly, eliminating, reducing, and diminishing bacteria, viruses and other harmful microbes is a major concern for all individuals as well as for the owners and operators of hotels, restaurants, arenas and other public gathering places. Bacteria and viruses may cause sicknesses, such as a cold, flu, viral outbreaks, irritation or exasperation of existing allergies, asthma and other breathing related issues, if not properly addressed.

In many cases, to maintain a properly sterilized and sanitized environment, manual labor is typically required by individuals and may be supplemented by robotic devices. For example, individuals spend a considerable amount of time cleaning and disinfecting a home, an office, a school, a business, public spaces or other areas. Manually cleaning every surface is time consuming, tiring, ineffective, and may result in some spots or areas being left unclean and untreated. This can lead to the spread of germs, bacteria, and dangerous viruses. Stated differently, it is difficult to ensure a high-level disinfection using manual cleaning and sanitizing efforts because of the amount of time and effort involved, the possibility of missed surfaces, re-contamination from dirty sponges, mops, rags and the like, and the improper use and mixing of antibacterial cleaning solutions.

By way of background, airborne bacteria, germs and viruses are oftentimes controlled through the use of sprays, filters, ultraviolet light emitters and/or air cleaning devices. For example, filters within heating, ventilation, and air conditioning (HVAC) systems may include chemical treatments and other electrostatic emitting devices to reduce the concentration of airborne bacteria, germs and viruses in the airflow being processed by such systems. Specially designed filtered devices may also be placed on a floor or a table and help to filter and recirculate air being treated through the filter device, or may be part of a central or forced air system that provides heating and cooling for a home, business or other enclosed space. Furthermore, sprays have been developed for use with a handheld aerosol canister and may be sprayed over a limited area for a short period of time, but are typically only useful when treating the surface area in the immediate vicinity of where the user and can are located.

Therefore, there exists a long felt need in the art for a portable and reusable disinfectant product that can be employed within homes, businesses, restaurants, schools, hospitals, and other enclosed area quickly, effectively and with minimal risk. There is also a long felt need in the art for a disinfectant fogger that emits a disinfecting spray designed to completely disinfect the enclosed space in which it is discharged into, and that effectively eliminates unpleasant odors, bacteria, germs, and viruses from the surroundings. More specifically, there is a long felt need in the art for a disinfectant fogger that allows the spray to permeate an entire room, covering all surfaces and hard to reach areas that may be missed by a user wielding a handheld device, and that enables users to maintain proper sanitary conditions within a specific location with a consistent mist droplet size and dispersal rate. Further, there is a long felt need in the art for a method of using a disinfectant product that requires minimal effort or input from the user, eliminates user error, and that is safe for residential use where both people and pets may cohabitate. Finally, there is a long felt need in the art for a disinfectant product that is relatively inexpensive to manufacture, is exceedingly effective, and that is safe and easy to use.

The subject matter disclosed and claimed herein, in one embodiment thereof, comprises a disinfectant fogger device comprised of an aerosol canister that contains a disinfectant solution, an opening in the aerosol cannister for releasing the disinfectant solution in a controlled and effective manner, a lock top that is activated by twisting and pushing downwards on the canister thereby forming a push and twist nozzle to release the disinfectant solution in the form of a mist. More specifically, the disinfectant fogger device of the present invention allows a user to simply press and twist the nozzle or activator to release the disinfectant solution from the aerosol canister and into the surrounding area. Additionally, the disinfectant solution of the disinfectant fogger device of the present invention is also both aesthetically pleasing (i.e., does not emit a foul or obnoxious odor) and cost effective, and the disinfecting device is relatively safe to use, as the disinfectant solution is only released upon the intentional activation of the nozzle mechanism (i.e., relatively childproof). The disinfectant fogger device also requires only minimal time and effort by the user to be activated and utilized.

In an alternate embodiment, the disinfectant fogger device may be activated by simply depressing a tab component that is immediately adjacent to the aerosol canister nozzle, which, in turn, both depresses the canister nozzle and holds it open so that the disinfectant solution may be continuously discharged from the aerosol canister in a mist like state via the cannister nozzle. A user may stop the discharge of the disinfectant solution from the aerosol cannister at any time by simply depressing the tab component again, which cases the cannister nozzle to close.

In this manner, the disinfectant fogger device of the present invention accomplishes all of the forgoing objectives, and provides a relatively safe, easy, convenient and cost-effective solution to disinfecting an entire enclosed space with a single action. More specifically, the disinfectant fogger device emits a disinfecting solution that is designed to completely disinfect the enclosed space (i.e., even hard to reach spaces and surfaces) in which it is discharged into, and effectively eliminates unpleasant odors, bacteria, germs, and viruses from the surroundings. The disinfectant fogger device also emits a consistent mist droplet size and dispersal rate, and requires minimal effort, input or time commitment from the user, thereby eliminating the possibility of use error.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a portable disinfectant fogger device comprised of an aerosol container, a unique and novel disinfectant solution contained or housed within said aerosol container, a disinfectant spray opening in the aerosol cannister that releases the disinfectant solution when activated, a lock top that is activated by twisting and pushing downwards on the aerosol container thereby forming a push and twist fogging nozzle capable of releasing the disinfectant solution in a controlled and consistent manner. More specifically, the disinfectant solution is stored in the aerosol container under pressure, wherein the internal pressure of the aerosol cannister causes the disinfectant solution to be dispersed in a consistent manner once the nozzle on the aerosol cannister is depressed and twisted in either a clockwise or counterclockwise direction.

The disinfectant fogger device of the present invention is portable, relatively lightweight and comprises a heavy duty metal or rigid plastic nozzle that does not wear out over time, and does not cause inconsistent mist droplet size. The nozzle may also include a screen element to maintain consistent droplet size distribution. The disinfectant solution used in the disinfectant fogging device may also be viricidal and/or be classified as a sporicidal disinfectant, meaning that it is capable of killing spore type organisms including, without limitation, C. diff, E. coli, etc.

In a further embodiment of the present invention, a method of disinfecting a room or other enclosed space to remove bacteria, germs, viruses and other microbiological contamination is disclosed, and includes the steps of: (a) initially providing an aerosol container containing a disinfectant solution in a liquid form and under relatively high pressure, such that the liquid disinfectant solution transforms to a gas at ambient room temperatures once the pressure is released from the aerosol container; (b) placing the aerosol container in an upright position at a desired location in the enclosed space (e.g., a room, a hall or other enclosed area); (c) pressing a nozzle present on the top of the aerosol container in the direction of the aerosol container; (d) twisting the nozzle in a clockwise or counter-clockwise direction while it is depressed; and (e) releasing the disinfectant solution from the aerosol container via the nozzle in a generally upward and 360 degree direction until either the desired amount of the disinfectant solution has been discharged or a predesignated amount of time has elapsed. The disinfectant fogger device of the present invention creates a uniform spray pattern throughout an enclosed space or room, thereby reaching otherwise hard to reach surfaces and spaces.

In a further embodiment of the present invention, a disinfectant fogger device is disclosed for dispensing a disinfectant solution to the surrounding area in an enclosed space. The disinfectant fogger device is comprised of an aerosol container configured to contain a disinfectant solution that is stored at a higher pressure than the ambient pressure, a diffusing nozzle coupled to the aerosol container to release the disinfectant solution in a fog or mist like emission through the diffusing nozzle when the nozzle is pressed down towards the aerosol container and twisted or rotated in a clockwise or counterclockwise direction. The emission is preferably directed from the nozzle of the aerosol canister in an upward and 360 degree direction so as to completely disinfect the enclosed space. More specifically, the high pressure of the disinfectant container is predetermined and is configured for a conventional space so that the fog of disinfecting agent disinfects the pathogens in the air and surfaces of the enclosed space.

In a still further embodiment of the present invention, a disinfectant fogger device may be provided which comprises a specific disinfectant formulation to address a particular need, such as decontaminating an area for a particular virus, bacteria or other microbial concern. In this manner, the disinfectant fogger device can be more effective in targeting the specific concern that gave rise to the need for its the use. For example, the disinfectant solution may contain a specific anti-viral or sanitizing solution comprising, for example, alcohol (80-100 proof), hydrogen peroxide (3-5% in solution), vinegar, essential oils and combinations of the same that can be used to sanitize an enclosed space of a particular virus, bacteria, germ or other microbial.

In a still further embodiment of the presently described invention, a disinfectant fogger device is disclosed for consumer use. The fogger device is comprised of an aerosol container that is constructed from a durable material to hold a disinfectant solution under pressure. The aerosol container is generally cylindrical in shape and is comprised of an interior space, side walls, a top portion and a bottom, wherein the top portion further comprises a nozzle that is movable from a first position to a second position. The nozzle further comprises a screen and a plurality of openings, wherein said openings are in fluid communication with the interior space when the nozzle is in the second position. The nozzle screen helps to control the disinfectant solution droplet size, with the preferable droplet size ranging from about 5 microns to about 50 microns, depending upon the particular application. The nozzle further has a predefined dispensing rate, and an effective active ingredient of between 10 to 90% alcohol by weight. The disinfecting solution is stored in the internal space under pressure when the nozzle is in the first position.

In yet a further embodiment of the presently described invention, a method of using the disinfectant fogger device of the present invention is disclosed and includes the steps of initially providing an aerosol container that is constructed from a durable material to hold a disinfectant solution under pressure. The container has an internal space, side walls, a top portion and a bottom, wherein the top portion further comprises a nozzle that is movable from a first position to a second position. The nozzle is further comprised of a screen to control the droplet size of the disinfectant solution as it is discharged from the internal space through the nozzle when the nozzle is in the second position. Next, the internal space of the aerosol container is filled with a disinfectant or sanitizing solution, and the solution is pressurized within the aerosol container. Then, the aerosol container is placed within an enclosed space that needs disinfecting or sanitizing, preferably in an upright manner. A user may then reposition the nozzle from the first position to the second position so that the disinfectant solution may be released into the enclosed space via the nozzle and the screen. The disinfectant solution preferably has a droplet size ranging from about 5 microns to about 50 microns, and has a predefined dispensing rate. The disinfectant solution is preferably comprised of between 10 to 90% active ingredient by weight.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one possible embodiment of the portable disinfectant fogger device of the present invention in accordance with the disclosed structure in an unused state;

FIG. 2 illustrates a perspective view of one possible embodiment of the portable disinfectant fogger device of the present invention having a tab in accordance with the disclosed structure, and a close up perspective view of the tab being depressed to activate the device;

FIG. 3 illustrates a perspective view of one possible embodiment of the portable disinfectant fogger device of the present invention placed in the middle of a room in accordance with the disclosed structure and discharging a disinfecting solution therein;

FIG. 4 illustrates a top perspective view of one possible embodiment of the portable disinfectant fogger device of the present invention in accordance with the disclosed structure; and

FIG. 5 illustrates a front perspective and cut-away view of the portable disinfectant fogger device of the present invention having a string loaded nozzle in accordance with the disclosed structure.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices may be shown in block diagram form in order to facilitate a description thereof. Various embodiments are discussed hereinafter. It should be noted that the figures are described only to facilitate the description of the embodiments. They are not intended as an exhaustive description of the invention or do not limit the scope of the invention. Additionally, an illustrated embodiment need not have all the aspects or advantages shown. Thus, in other embodiments, any of the features described herein from different embodiments may be combined.

As noted above, there is a long felt need in the art for a portable and reusable disinfectant device that can be utilized in an enclosed space quickly, effectively and with minimal risk. There is also a long felt need for a disinfectant fogger device that emits a disinfecting spray designed to completely disinfect the enclosed space in which it is discharged into, and that effectively eliminates unpleasant odors, bacteria, germs, and viruses from the surroundings. Further, there is a long felt need in the art for a method of using a disinfectant fogger device that requires minimal effort or input from the user, eliminates or reduces the possibility of user error, and that is safe for residential use by non-experts.

The present invention, in one exemplary embodiment, is a portable disinfectant fogger device that comprises an aerosol container that contains one of a disinfectant or sanitizing solution, a spray opening that releases the solution on demand, a lock top having a nozzle that is activated by pushing downwards on the nozzle and twisting the same in a clockwise or counter-clockwise direction. The disinfectant or sanitizing solution is contained within the aerosol container under pressure, and is released once the nozzle activated. The disinfectant fogger device of the present invention is easily portable, and has heavy duty metal or plastic nozzle that does not wear out over time and does not cause inconsistent mist droplet size or discharge rates.

Droplet sizes ranging between 5-50 microns (μm) in diameter and emanating from the disinfectant fogger device of the present invention have proven to be highly effective, and droplet sizes of between 10 and 35 microns in diameter are preferable. More specifically, droplets of these sizes are ideal for combatting pathogens, vector carriers, pests and the like. In addition, in a preferred embodiment, the disinfectant solution is comprised of an active ingredient concentration ranging from 10-90% by weight, with the remaining portions making up propellants, stabilizers, and/or other non-treatment components, and at flow rates of up to 0.52 quarts per minute (31.7 quarts per hour or nearly 8 gallons an hour). The application of disinfectants, sanitizing solutions and biocides via aerosol or fogging can significantly reduce the number of viable infectious pathogens in a particular area. More specifically, foggers produce micro droplets that float or stay suspended in the air for approximately 10 minutes after application, thereby enabling the droplets to reach the most inaccessible portions of the enclosed space where conventional cleaning or spraying can't reach. The smaller the droplets the longer the disinfecting particles will remain airborne, and it is believed that approximately one ounce of the disinfectant solution of the present invention will typically cover around 1,000 cubic feet.

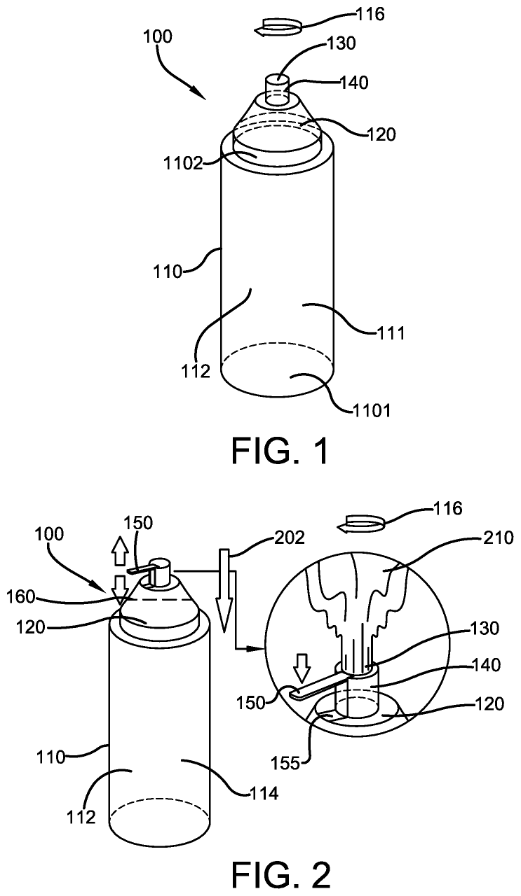

Referring initially to the drawings, FIG. 1 illustrates a perspective view of one possible embodiment of a portable disinfectant fogger device 100 of the present invention in accordance with the disclosed structure. The portable disinfectant fogger device 100 comprises a generally cylindrical shaped aerosol container 110 that houses an aerosol solution 112 therein under pressure, wherein the pressure inside the aerosol container 110 is higher than the ambient pressure of the enclosed space in which the disinfectant fogger device 100 will be used. The aerosol canister or container 110 is generally constructed from steel or other durable material in order to hold the contents under pressure. However, it is also contemplated that other suitable materials could also be used depending on the amount of pressure and the disinfectant solution stored therein.

The cylindrical shaped container 110 is comprised of a closed bottom end 1101, and an open top end 1102. The container 110 may be of any conventional design, and is positionable in an upright position on a floor, a table or any similar horizontal surface at a desired location within the enclosed space, such as in a middle of a room. The container or canister 110 may also be placed on a stool, a chair or the like so that heavier furniture, such as a table, does not have to be moved in order to disperse the contents of the container 110 in the center of the enclosed space. The aerosol solution 112 may be a pressurized aerosol of any conventional design, and a chemical disinfectant or sanitizer 114 may be a gaseous disinfectant, sanitizer, an atomized fluid disinfectant and/or any other chemical disinfectant or sanitizer that can be delivered via an aerosol 112 to an enclosed space. Moreover, the chemical solution 114 may have anti-bacterial and/or anti-viral properties.

The disinfectant fogger device 100 is also comprised of a nozzle 120, wherein the nozzle 120 may further comprise one or more of the following: an orifice 130, a nozzle screen 135, a nozzle button 140, a nozzle tab 150, a nozzle tab lock 155, and a nozzle seal 160. The nozzle 120 is coupled to the aerosol container 110 at the open end 1102, and is repositionable between a first position (e.g., closed position) and a second position (e.g., an open position), or vice versa. The nozzle 120 is in fluid communication with an interior of the aerosol container 110 when it is in the second position, but not the first position. To reposition the nozzle 120 from the first position to the second position, a user (not shown) would press down on the nozzle 120 (i.e., in the direction of container 110) and twist the same in a desired direction such as in a clockwise or counterclockwise direction 116, thereby opening a valve present underneath the nozzle button 140 and facilitating the aerosol 112 and the chemical solution 114 to be released from the container 110 through the nozzle opening/orifice 130 and into the enclosed space. In this manner, the chemical solution 114 is distributed evenly throughout the enclosed space to disinfect and/or sanitize the entirety of the same. The enclosed space may be, for example, a room that has been exposed to an infectious disease, a virus, a bacteria or other undesirable microbe.

FIG. 2 illustrates a perspective view of one potential embodiment of a nozzle of the portable disinfectant fogger device of the present invention in accordance with the disclosed structure. The disinfectant fogger device 100, when activated, causes a disinfectant or sanitizing liquid, solution or gas 114 under pressure to be dispensed through an outlet nozzle 120. As best shown in FIG. 2, to release the solution 114 and distribute it comprehensively and evenly throughout an enclosed space such as a room, the user depresses the nozzle 120 in a generally downward direction 202 towards the container 110 and twists the same in a generally circular motion 116, such as in a clockwise or counterclockwise direction, by operation of the rotating assembly of the nozzle 120. More specifically, when the nozzle 120 is pressed downwards, a valve present at the top of the container 110 (and just underneath the nozzle button 140) has a spring to prevent it from staying permanently open. During the pressing down of the nozzle 120, the force exerted by the user is greater than the bias in the spring, thereby enabling the valve to open. When the valve is opened, the pressure within the container 110 causes the aerosol 112 and disinfectant 114 to escape through the orifice 130 as a dispersing spray 210. After use, when the nozzle 120 is released, the spring causes the valve to close again, thereby preventing the aerosol 112 and the disinfecting solution 114 to further escape from the aerosol container 110.

Alternatively, and as also shown in FIG. 2, a tab 150 may be provided which when depressed unseats the nozzle 120 from its seated position via a locking tab element 155. When the tab 150 is depressed again the locking tab element unseats itself, thereby allowing the nozzle 120 to return to its original closed condition. In another embodiment, the tab 150 may simply unseat the nozzle 120 from its locked position, breaking a frangible seal 160 and allowing the aerosol/disinfecting solution 112/114 to be dispersed to the room. The frangible seal 160 may be made of aluminum, plastic or other film sufficiently strong to withstand the pressure of the contents from pre-mature release.

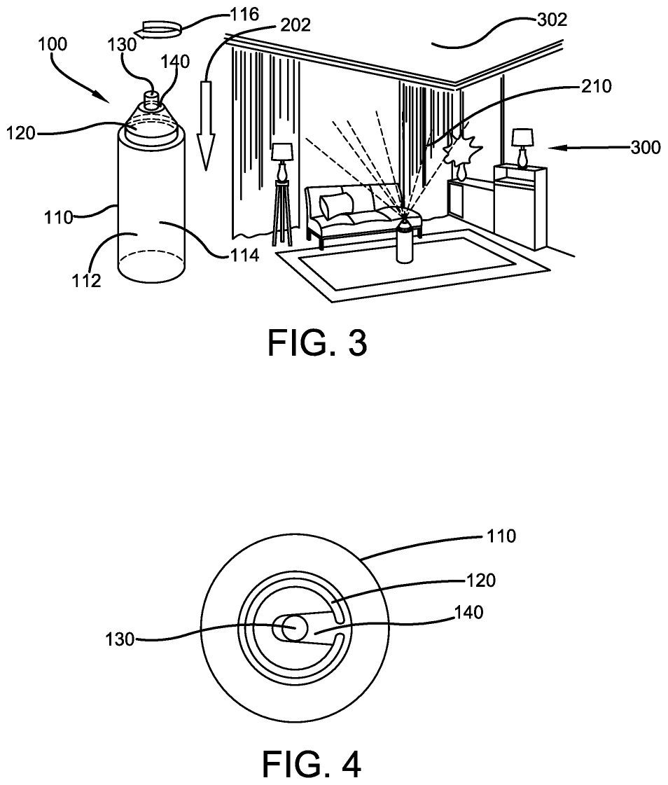

FIG. 3 illustrates a perspective view of one potential embodiment of the portable disinfectant or sanitizing fogger device 100 of the present invention placed in the middle of a room in accordance with the disclosed structure. In use, the fogger device 100 may be positioned on a floor near a middle of the room 300 when the room 300 needs to be disinfected or sanitized due to exposure to an infectious disease or any other conceivable reason why the room 300 would need to be disinfected or sanitized. The user may then activate the fogger device 100 by pressing downwards 202 on the nozzle and rotating it in a first direction 116 to open and lock the valve. The opening of the valve allows the pressurized aerosol 112 contained therein that contains the disinfectant solution 114 to be released from the container 110 and evenly diffused throughout the room 300. In this manner, the chemical solution 114 in the aerosol 112 contacts all of the various surfaces, walls and ceiling, including the furniture and other item in the room 300, and disinfects and/or sanitizes the room 300 without requiring one or more individuals to manually disinfect or sanitize the room 300. Once the device 100 is activated (which may be on a small time delay), the room 300 is preferably left closed or otherwise sealed off for a selected amount of time to allow the chemical solution 114 to fully disinfect or sanitize the room 300. More specifically, the spray 210 of aerosol 112 and the chemical solution 114 is propelled outwardly and upwardly to a height near or at the ceiling 302 of the room 300. The spray droplets 210 then fall downward due to gravity and impinge on succeeding upwardly propelled spray droplets 210. The net effect is a mushrooming of the spray droplets 210 throughout the entire room, descending to contact all exposed surfaces. The droplets 210 are intended to be small enough in size so that any furniture or other surfaces are not left with a sticky or wet feeling once individuals reenter the room or area where the container or canister 100 was deployed.

FIG. 4 illustrates a perspective top view of one possible embodiment of the portable disinfectant fogger product 100 of the present invention in use in accordance with the disclosed structure. As shown, the nozzle button/flap 140 may be used to press the nozzle 120 downwardly onto the container 110, which unlocks the nozzle 120 by opening the valve to enable the pressurized sanitizing/disinfectant liquid 114 and aerosol 112 to be emitted as an aerosol spray 210 through a spray opening or orifice 130. In a preferred embodiment, the spray orifice 130 is designed to provide for a generally 360-degree fog-type emission that occurs in both an upward and outward manner in relation to the cannister 110. As also shown in FIG. 4, the nozzle screen 135 can be used to produce a desired droplet size, and prevent disproportionate discharge of the chemical solution 114 from leaving the cannister or container 110.

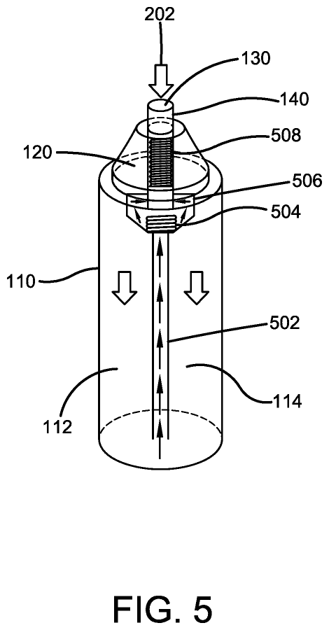

FIG. 5 illustrates a perspective front and cut-away view of one potential embodiment of the portable disinfectant fogger device 100 of the present invention in accordance with the disclosed structure. As shown, when the nozzle button 140 of the nozzle 120 is pressed by a user in a downward direction 202, an upper frame 508 present in the nozzle 120 is pressed downwards and, as a result, spring 504 is tightly compressed and the valve 506 is permitted to open. As a result, the pressurized aerosol 112 and disinfectant 114 solution is directed upwardly through the tube/pipe 502, into the valve 506 and then the upper frame 508 area, and eventually discharged through the opening 130 in nozzle 120.

In an alternate embodiment of the present invention, the disinfectant fogger device 100 may further comprise a timing controller (not shown) and/or a sensor module (also not shown) having one or more status sensors, such as motion, heat sources, light and/or other sensors, that prevents the device 100 from initiating while the sensors detect activity in the enclosed space to prevent a user from coming into contact with, or inhaling, the disinfecting solution 114. If the sensor module senses conditions that are not suitable or ideal for disinfection of the enclosed space the disinfectant defogger device 100 may not permit the release of the disinfectant spray 114 into the surroundings. Further, the timing controller can be programmed with a specific time duration as desired by the user to automatically release the disinfectant solution 114 into the surroundings for the specified duration.

The aerosol container 110 may be made available in various different sizes and colors to accommodate the particular wants and needs of the user. Exact size, measurement, construction and design specifications of the unique fogger device 100 of the present invention may vary upon manufacturing and/or the particular materials used. Additionally, the aerosol container 110 may also have a name tag, name badges, laser-graving, customizable colors and fonts, embroidery and prints.

In one embodiment, the aerosol container 110 may comprise 8 to 20 ounces of the disinfectant solution 114, which is preferably a n-decyl dimethyl ammonium chloride in an alcohol diluent. It may also contain an additional fragrance. The aerosol 112 is preferably a compressed gas such as pentane, propane or butane. In a preferred embodiment, the expelled liquid droplets 210 are generally benign to all surfaces that they come into contact with, whether made of fabric, wood, paint, paper, etc., and will not stain the same. They will, however, disinfect and sanitize said surfaces. It is anticipated that the entire dispersal can be accomplished approximately 2-2½ minutes, with the droplets 210 drying upon contact. Thereafter, the room 300 may be entered to collect and potentially dispose of the fogger device 100.

In other embodiments, the disinfectant solution 114 emitted from the aerosol container 110 may include an ammonium compound such as alkyl dimethylbenzyl ammonium saccharinate in the amount of approximately 1 percent. The disinfecting solution 114 may also include ethanol for a quick-drying application of 75 percent of the total formula by weight. The disinfectant spray according to the present invention kills up to 99.9 percent of all germs existing in the air space in which the solution is emitted. The disinfecting substance 114 may be a room temperature (e.g., 20° C. to 25° C.) substance that can be dispersed as a fog during operation. In other embodiments, the disinfecting substance 114 may be used at temperatures in the range of between about −40° C. to 100° C. In some embodiments, the disinfecting substance 114 includes a peracetic acid, a hydrogen peroxide (H₂O₂), a hospital grade disinfectant, and/or an antimicrobial solution.

The disinfectant fogger device 100 may further comprise a hypoallergenic disinfecting, antibacterial and antiviral solution which is specially formulated to kill germs, virus and bacteria on contact. In an embodiment, the disinfectant fogger formula is a quick drying solution and will dry completely within ten seconds of application to a designated surface. The device 100 may further comprise a generally cylindrically shaped cap to cover the nozzle between uses. It is also contemplated that fogger device described herein would also be suitable for use in dispensing insecticides, pesticides, or other propellants.

Use of the portable disinfectant fogger device 100 of the present invention is very simple, and requires little user input, effort or time. Nonetheless, another productive way to use the portable disinfectant fogger device 100 is to insert it into an optional wall mounted housing unit. This wall mounted housing unit can be fabricated of plastic, stainless steel or other suitable materials and mounted to the wall via one or more mounting brackets. In use, the wall mounted housing unit will retain the disinfectant fogger device 100 while its pressurized contents are discharged into the room.

As previously stated, the disinfectant fogger device 100 of the present invention provides a means to evenly distribute a disinfectant solution 114 throughout an enclosed area and thus mitigate odors, eliminate fungi, and destroy up to 99.9 percent of the bacteria, viruses, germs and other undesirable microbes in the air or on a surface in an enclosed space. The orifice 130 according to the present invention provides for a generally vertical, 360-degree propulsion of the aerosol and disinfecting solution 112, 114 to create a fog like mist 210 within the enclosed room 300 where the disinfectant fogger device 100 is used. A child safety seal may also be inserted within the nozzle 120, and therefore prevents the unintentional use of the disinfectant spray 114 by children.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “portable disinfectant fogger product”, “disinfectant fogger”, “fogger device”, and “disinfectant fogger product” are interchangeable and refer to the portable disinfectant fogger product 100 of the present invention. It should also be noted that the nozzle(s) might spray (sprayer), mist (mister), and disperse (disperser). The terms spray, mist, and disperse are used as examples throughout the specification and claims, however, embodiments described as spraying may also be misting and/or dispersing. Similarly, embodiments described as misting may also be spraying and/or dispersing and embodiments described as dispersing may also be misting and/or spraying.

Notwithstanding the forgoing, the portable disinfectant fogger product 100 of the present invention can be of any suitable size and configuration as is known in the art without affecting the overall concept of the invention, provided that it accomplishes the above stated objectives. One of ordinary skill in the art will appreciate that the size, configuration and material of the portable disinfectant fogger product 100 as shown in the FIGS. are for illustrative purposes only, and that many other sizes of the portable disinfectant fogger product 100 are well within the scope of the present disclosure. Although the dimensions of the portable disinfectant fogger product 100 are important design parameters for user convenience, the portable disinfectant fogger product 100 may be of any size that ensures optimal performance during use and/or that suits pet owners need and/or preference.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof. For example, while the disinfectant fogger device 100 has been generally described as a portable cylinder-like assembly, it will be appreciated that other configurations are also possible, such as ceiling mounted, door mounted, or wall mounted configurations.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A disinfectant fogger device for use in an enclosed space and comprising;
a container constructed from metal and formed into a generally cylindrical shape, the container having a top component, a side wall and a bottom, wherein the top component further comprises a nozzle moveable from a first closed position to a second open position;
a solution selected from one of a disinfecting or a sanitizing formulation, the solution provided in the container and held under a pressure that is greater than an ambient pressure of the enclosed space; and
the container and the nozzle producing a solution droplet size ranging from about 5 microns to about 50 microns in diameter, having a predefined solution dispensing rate and the solution having an active ingredient concentration of between 10 to 90% by weight.

2. The disinfectant fogger device of claim 1, wherein the solution droplet size is between 10 and 35 microns in diameter.

3. The disinfectant fogger device of claim 1, wherein the predefined solution dispensing rate is between 30 to 80% per minute.

4. The disinfectant fogger device of claim 1, wherein the solution comprises an ethanol.

5. The disinfectant fogger device of claim 1, wherein the solution is the sanitizing formulation.

6. The disinfectant fogger device of claim 5, wherein the sanitizing formulation is comprised of an alcohol, a hydrogen peroxide, a vinegar, and an essential oil.

7. The disinfectant fogger device of claim 1, wherein the solution is the disinfecting formulation.

8. The disinfectant fogger device of claim 7, wherein the disinfecting formulation is comprised of a peracetic acid, a hydrogen peroxide, a disinfectant, and an antimicrobial solution.

9. The disinfectant fogger device of claim 7, wherein the disinfecting formulation is comprised of an ethanol.

10. The disinfectant fogger device of claim 7, wherein the disinfecting formulation kills 99.9% of a germ and a bacteria in an application zone.

11. The disinfectant fogger device of claim 1, wherein the nozzle comprises a frangible seal and a screen.

12. The disinfectant fogger device of claim 1, wherein the nozzle comprises a spring biased valve held within an upper nozzle frame of the container.

13. A disinfectant fogger device comprising:
a container, wherein the container comprises a side wall, a top, a bottom, and a nozzle that is movable from a first position to a second position; and
a solution contained under pressure within the container, the solution including one of a disinfecting solution or a sanitizing solution and comprising an active ingredient concentration of between 10 and 90% by weight,
wherein the nozzle further comprises a screen to maintain a droplet size of the solution ranging from about 5 microns to about 50 microns in diameter, and the nozzle having a predefined solution dispensing rate.

14. The disinfectant fogger device of claim 13, wherein the solution is comprised of an alcohol having a proof of between 80 and 100, a hydrogen peroxide, a vinegar, and an essential oil.

15. The disinfectant fogger device of claim 13, wherein the solution is the disinfecting formulation.

16. The disinfectant fogger device of claim 13, wherein the solution is comprised of a peracetic acid, a hydrogen peroxide, a disinfectant, and an antimicrobial.

17. The disinfectant fogger device of claim 16 further comprising an ethanol.

18. The disinfectant fogger device of claim 13, wherein the nozzle comprises a spring biased valve held within an upper nozzle frame of the container.

19. A method of using a disinfectant fogger device, the method comprising the steps of;
providing a container constructed to hold a solution under pressure, the container having a side wall, a top and a bottom, wherein the top further comprises a nozzle that is repositionable from a first position to a second position, and further wherein the nozzle comprises a screen;
filling the container with the solution, wherein the solution is at least one of a disinfecting solution and a sanitizing solution;
pressurizing the container with the solution contained therein to a pressure that is greater than an ambient pressure outside of the container;
placing the container within an enclosed space for treatment;
repositioning the nozzle from the first position to the second position; and
releasing the solution into the enclosed space, wherein the solution is released having a droplet size ranging from about 5 microns to about 50 microns in diameter.

20. The method of using a disinfectant fogger device of claim 19, wherein the solution is comprised of a peracetic acid, a hydrogen peroxide, a disinfectant, and an antimicrobial.
